(19) United States
(12) Patent Application Publication
(10) Pub. No.: US 2007/0225730 A1
(43) Pub. Date: Sep. 27, 2007

(54) Iris basket

(76) Inventor: Travis Deal, Freedom, IN (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/384,519

(22) Filed: Mar. 21, 2006

(51) Int. Cl. A61B 17/22

(52) U.S. Cl. 606/127

(57) ABSTRACT

A medical device includes a sheath defining a lumen, an elongate member disposed within the lumen, and a basket including a plurality of legs connected to the elongate member. A distal end of each of the plurality of legs moves along a length of an adjacent one of the plurality of legs.

### FIELD OF THE INVENTION

This disclosure relates generally to medical devices and, more particularly, to devices for the capture and/or retrieval of objects within a body, such as stones, other calculi, or foreign matter, and methods of using the same.

### BACKGROUND OF THE INVENTION

Extractors have been used to remove stones and other calculi from within the body of a patient. One type of extractor includes a sheath, an elongate member extending within and moveable relative to the sheath, and a basket connected to the distal end of the elongate member. The basket may have a number of legs, and may have a collapsed position within the sheath and an expanded position beyond a distal end of the sheath. When the basket is transitioned from the collapsed position to the expanded position, the basket may extend beyond the distal end of the sheath in a substantially distal direction. The basket may also extend distal the targeted stone. The stone may then be captured within the expanded basket and removed from the body.

When removing a targeted stone from complex, tortuous body structures, however, it may be difficult to capture the stone within such baskets. In particular, it may be difficult to extend such baskets in the distal direction to capture the stone, as doing so may push the stone to a further distal position within the body structure. In addition, extending such baskets in the distal direction may cause damage or trauma to the surrounding body tissue.

The present disclosure provides retrieval devices and methods of using the same that avoid some or all of the aforementioned shortcomings of existing devices.

### SUMMARY OF THE INVENTION

In accordance with an exemplary embodiment of the present disclosure, a medical device includes a sheath defining a lumen, an elongate member disposed within the lumen, and a basket including a plurality of legs connected to the elongate member. A distal end of each of the plurality of legs moves along a length of an adjacent one of the plurality of legs.

In accordance with certain embodiments of the present disclosure, the device may include one or more of the following features. For example, at least one of the plurality of legs includes a joint fixedly connected to the distal end of the leg, the joint is configured to movably connect the at least one leg to the adjacent one of the plurality of legs, and the basket transitions from a collapsed position to an expanded position in a substantially radial direction. In addition, the distal end of at least one of the plurality of legs moves in one of a substantially counter-clockwise direction and a substantially clockwise direction about a longitudinal axis of the sheath, as viewed from an open end of the basket, when the basket is transitioned from a collapsed position to an expanded position.

In other exemplary embodiments, a distal end of the basket forms a predetermined shape in an expanded position. The predetermined shape is one of substantially triangular, substantially square, and substantially round. In addition, at least one of the plurality of legs includes a first section, a second section, and a third section, and the first section extends substantially parallel to a longitudinal axis of the sheath when the basket is in an expanded position. The second section is disposed at a pitch angle when the basket is in an expanded position, and the pitch angle is defined by the second section and a plane perpendicular to the longitudinal axis of the sheath. The second section is curved when the basket is in an expanded position, and the second section defines a width of the basket when the basket is in an expanded position. Moreover, the third section extends substantially perpendicular to the longitudinal axis of the sheath when the basket is in an expanded position.

In still other exemplary embodiments, a distal end of the basket is substantially open when the basket is in an expanded position, the distal end of the basket is tipless, and the basket comprises at least three legs. In addition, the device further includes a guide connected to a distal end of the sheath, and at least one of the plurality of legs is movably disposed within a corresponding lumen of the guide. Moreover, the plurality of legs define a height and a width of the basket. The basket is configured such that the width of the basket remains substantially constant and the height of the basket decreases during at least an initial portion of the basket transitioning from an expanded position to a collapsed position.

In accordance with another exemplary embodiment of the present disclosure, a medical device includes a basket including a plurality of legs defining a height and a width of the basket. The basket is configured such that the width of the basket remains substantially constant and the height of the basket decreases during at least an initial portion of the basket transitioning from an expanded position to a collapsed position.

In accordance with certain embodiments of the present disclosure, the device may include one or more of the following features. For example, the device further includes a sheath defining a lumen. At least one of the plurality of legs extends substantially completely beyond a distal end of the sheath in the expanded position, and the at least one leg is disposed substantially completely within the lumen of the sheath in the collapsed position. A distal end of the at least one leg moves in one of a substantially counter-clockwise direction and a substantially clockwise direction about a longitudinal axis of the sheath, as viewed from an open end of the basket, when the basket is transitioned from the collapsed position to the expanded position. The device further includes a guide connected to the distal end of the sheath, and the at least one leg is movably disposed within a corresponding lumen of the guide.

In other exemplary embodiments, the least one leg includes a first section, a second section, and a third section. The first section extends substantially parallel to a longitudinal axis of the sheath when the basket is in an expanded position. The second section is disposed at a pitch angle when the basket is in an expanded position. The pitch angle is defined by the second section and a plane perpendicular to the longitudinal axis of the sheath. The second section is curved when the basket is in an expanded position, and the second section defines the width of the basket when the basket is in an expanded position. In addition, the third section extends substantially perpendicular to the longitudinal axis of the sheath when the basket is in an expanded position.

In further exemplary embodiments, a distal end of the basket is substantially open when the basket is in an expanded position, the distal end of the basket is tipless, and the basket comprises at least three legs.

In accordance with yet another exemplary embodiment of the present disclosure, a method of removing matter from the body of a patient includes providing a medical device including a sheath defining a lumen, an elongate member disposed within the lumen, and a basket including a plurality of legs connected to the elongate member. A distal end of each of the plurality of legs moves along a length of an adjacent one of the plurality of legs. The method further includes advancing the medical device to a treatment site within the body of the patient, capturing the matter within the basket of the device, and removing the medical device from the body of the patient.

In accordance with certain embodiments of the present disclosure, the method may include one or more of the following features. For example, the matter is one of a kidney stone, a struvite, a uric acid stone, a cystine stone, and a solid deposit, and capturing the matter further includes reducing a height of the basket while maintaining a width of the basket substantially constant. In addition, the method further includes transitioning the basket from a collapsed position to an expanded position. Transitioning the basket further includes manipulating the distal end of at least one of the plurality of legs in one of a substantially counter-clockwise direction and a substantially clockwise direction about a longitudinal axis of the sheath, as viewed from an open end of the basket. Transitioning the basket also includes forming a distal end of the basket into a predetermined shape in the expanded position. The predetermined shape is one of substantially triangular, substantially square, and substantially round.

It is to be understood that both the foregoing general description and the following detailed description are exemplary and explanatory only, and are not restrictive of the invention as claimed.

### BRIEF DESCRIPTION OF THE DRAWINGS

FIG. 1 is a partial cross-sectional view of a retrieval device according to an exemplary embodiment of the present disclosure.

FIG. 2 illustrates an alternate configuration of the device of FIG. 1.

FIG. 3 is an end view of a basket according to an exemplary embodiment of the present disclosure.

FIG. 4 is a side view of a portion of a basket leg according to an exemplary embodiment of the present disclosure.

FIG. 5 is a plan view of the basket leg of FIG. 4.

### DETAILED DESCRIPTION

Exemplary embodiments of the present disclosure are illustrated in the accompanying drawings. Wherever possible, the same reference numbers will be used throughout the drawings to refer to the same or like parts.

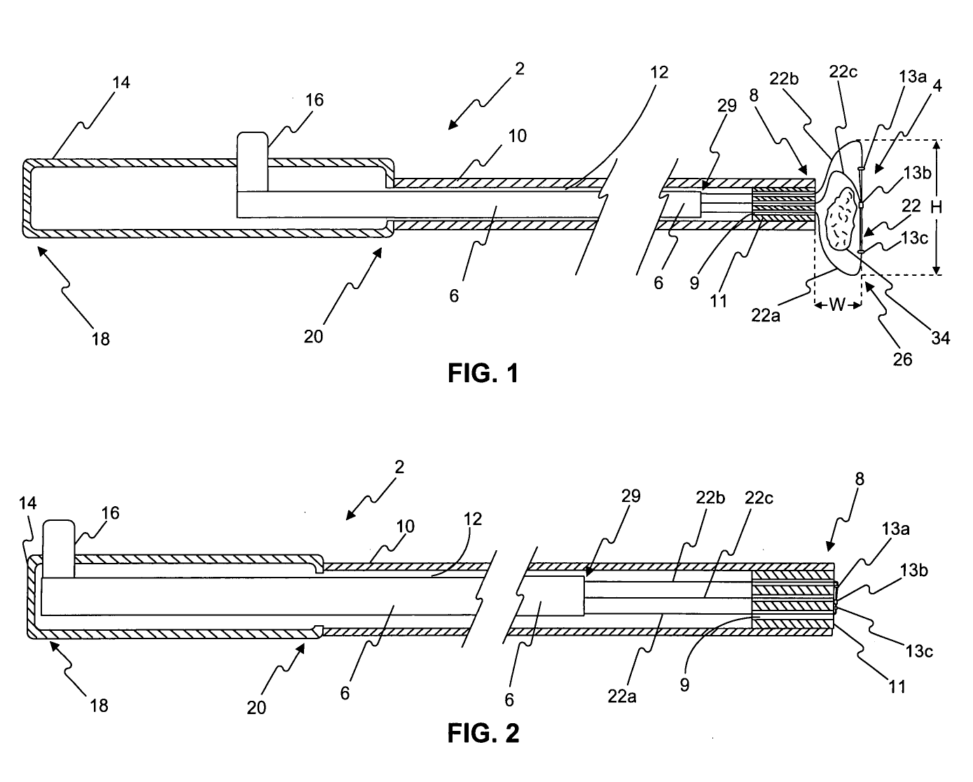

FIG. 1 illustrates a retrieval device 2 according to an exemplary embodiment of the present disclosure. The device 2 includes a basket 4 having a plurality of legs 22. The legs 22 may be connected to a distal end 29 of an elongate member 6. Although, for example, FIGS. 1-3 show a basket 4 having three legs 22a, 22b, 22c, other exemplary embodiments of the basket 4 may include more or fewer than three legs 22a, 22b, 22c, to facilitate the immobilization and/or retrieval of a stone 34, calculi, or other foreign matter. The device 2 further includes a sheath 10 defining at least one lumen 12, and the elongate member 6 is movably disposed within the lumen 12. Relative movement between the elongate member 6 and the sheath 10 assists the basket 4 in transitioning between an expanded position (shown in FIG. 1) where the basket 4 is disposed substantially beyond a distal end 8 of the sheath 10, and a collapsed position (shown in FIG. 2) where the basket 4 is disposed substantially within the lumen 12 of the sheath 10.

As shown in FIGS. 1 and 2, the device 2 may further include a handle 14 configured to assist in transitioning the basket 4 between the expanded position and the collapsed position. The handle 14 may include, for example, a thumb slide 16 or other conventional mechanisms configured to assist in manipulating the position of the basket 4. In an exemplary embodiment, moving the thumb slide 16 toward a distal end 20 of the handle 14 may move the elongate member 6 in a distal direction relative to the handle 14 and the sheath 10 and, thus, may assist in transitioning the basket 4 to the expanded position shown in FIG. 1. In such an embodiment, moving the thumb slide 16 toward a proximal end 18 of the handle 14 may move the elongate member 6 in a proximal direction relative to the handle 14 and the sheath 10 and, thus, assist in transitioning the basket 4 to the collapsed position shown in FIG. 2. In this exemplary embodiment, the sheath 10 may remain stationary with respect to the handle 14. In additional exemplary embodiments, however, the elongate member 6 and the basket 4 may remain stationary with respect to the handle 14, and the thumb slide 16 may be configured to move the sheath 10 relative to the basket 4.

The elongate member 6 may be formed from, for example, a wire, rod, tube, hypotube, cannula, stent, or other piece of biocompatible material or combination of biocompatible materials known in the art. Such materials may include, but are not limited to, polyamide, PEBAX, stainless steel (such as 300 and 400 series), cobalt chromium, nickel, titanium, nitinol, thermoforming plastic, polytetrafluoroethylene (“PTFE”), and expanded polytetrafluoroethylene (“ePTFE”). The elongate member 6 may also be a metal coated with a polymer and may have one or more layers of material. The elongate member 6 may be solid or hollow, and may be substantially cylindrical. Alternatively, the elongate member 6 may be formed from a flat sheet of material. If formed from a flat sheet, the elongate member 6 may be formed into a substantially cylindrical shape.

The overall length and diameter of the elongate member 6 may vary depending on the application. For example, a relatively long elongate member 6 may be advantageous for retrieving stones or other calculi deep within the body of the patient. In addition, an elongate member 6 having a relatively small diameter may be advantageous for retrieving stones from restricted passageways within the human urinary tract. The elongate member 6 may be relatively flexible to facilitate the retrieval of stones located in complex, tortuous body structures.

The elongate member 6 may be connected to the legs 22 in any conventional way. For example, in an embodiment of the present disclosure, the elongate member 6 and the legs 22 may be connected with adhesives, weldments, or solder. In an additional exemplary embodiment, the elongate member 6 may be formed from each leg 22. In such an embodiment, the legs 22 may be intertwined or braided to form the elongate member 6. Alternatively, the elongate member 6 may be comprised of unbraided legs 22 that are bonded, adhered, or otherwise connected together. In still another exemplary embodiment, the legs 22 may be cut, etched, stamped, and/or otherwise formed from the elongate member 6.

The sheath 10 may be formed from any of the materials discussed above with respect to the elongate member 6. Although FIGS. 1 and 2 illustrate a sheath 10 having a single lumen 12, in additional exemplary embodiments of the present disclosure, the sheath 10 may define more than one lumen 12. The sheath 10 may be dimensioned and/or otherwise configured to fully enclose the elongate member 6 and substantially the entire basket 4 when the basket 4 is in the collapsed position shown in FIG. 2.

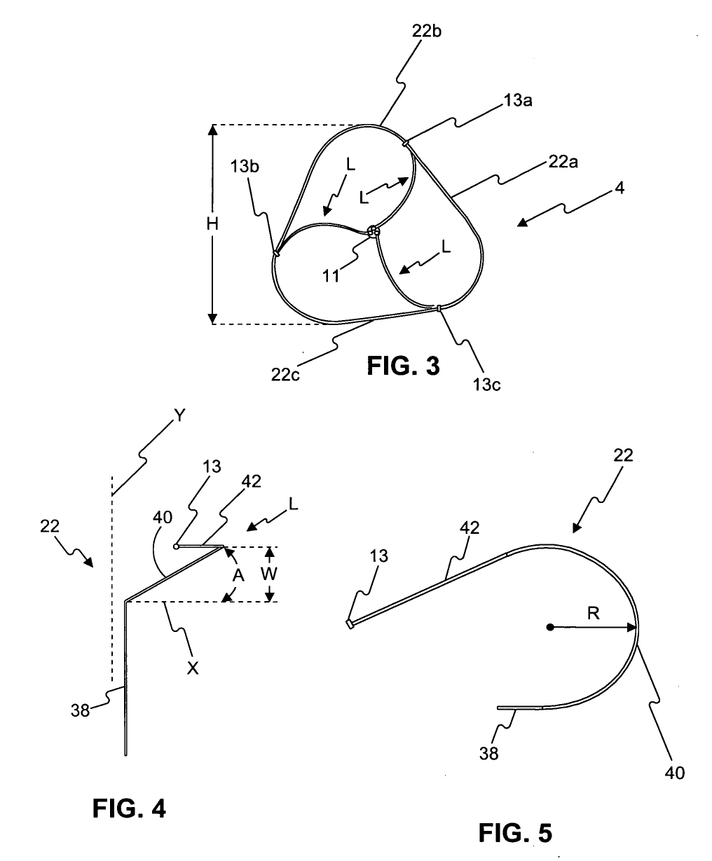

As shown in FIGS. 1-5, the legs 22 may be discrete wires, fibers, strands, filaments, or like elongate structures. The legs 22 may be made from any biocompatible metal or metal alloy, such as, for example, stainless steel, cobalt chromium, nickel, titanium, and nitinol. The legs 22 may include any cross-sectional shape known in the art, such as, for example, cylindrical, flat, square, semicircular, arced, D-shaped, rectangular, or ovular. Each leg 22 may be cold worked or heat processed to form a shape in memory. The shape may be fully formed when the basket 4 is fully expanded. Alternatively, the shape may be partially formed when the basket 4 is partially expanded. The resulting basket 4 may be any shape useful in immobilizing, capturing, and/or retrieving a stone. Such shapes may include, for example, substantially round, substantially square, and substantially triangular, as viewed from an open distal end 26 (FIG. 1) of the basket 4 (such as, for example, from the perspective of FIG. 3). In an exemplary embodiment, the shape of the basket 4 may be related to the number of legs 22 included in the basket 4, and the basket 4 may take on an increasingly round shape as additional legs 22 are included. For example, in such an embodiment, a fully expanded basket 4 having three legs 22 may be substantially triangular, a fully expanded basket 4 having four legs 22 may be substantially square, and a fully expanded basket 4 having five or more legs 22 may be substantially round.

As the basket 4 is transitioned from the collapsed position to the expanded position, the legs 22 may extend from the distal end 8 of the sheath 10 in a substantially radial direction relative to the sheath 10. As viewed from the open distal end 26 of an exemplary basket 4, (such as, for example, from the perspective of FIG. 3) the distal end of each leg 22 may move in a substantially counter-clockwise direction about the sheath 10 as the basket it transitioned from the collapsed position to the expanded position. When such a basket 4 is transitioned from the expanded position to the collapsed position, the distal ends of the legs 22 may move in a substantially clockwise direction when viewed from the same perspective. Alternatively, the distal ends of the legs 22 may move in a substantially clockwise direction about the sheath 10 when the basket 4 is transitioned from the collapsed position to the expanded position, and the distal ends may move in a substantially counter-clockwise direction when the basket 4 is collapsed. The exemplary basket 4 may, thus, expand and collapse in a substantially iris-like motion. Transitioning the basket 4 in this way may assist a user in capturing, immobilizing, and/or removing a stone 34 from within the body and may reduce the likelihood of pushing or otherwise moving the stone 34 in a distal direction as the basket 4 expands. Transitioning the basket 4 in this way may also assist in dilating, for example, the urinary tract, or other body structure in which the stone 34 is located without causing trauma thereto. Such dilation may further assist in capturing, immobilizing, and/or retrieving the stone 34. Moreover, transitioning the basket 4 as described above may result in a basket 4 with an open distal end. Such a basket 4 may be moved distally over the stone 34 and may then be collapsed around the stone 34 to grasp it between, for example, the legs 22.

In addition, the height H (FIG. 1 and 3) of such an exemplary basket 4 may increase as the basket 4 is expanded while the width W (FIGS. 1 and 4) of the basket 4 may increase minimally during the initial stages of expansion and may remain substantially constant during the later stages of expansion. The height H may also decrease as the basket 4 transitions from the expanded position to the collapsed position, while the width W may remain substantially constant until the later stages of retraction. Thus, a stone 34 may be immobilized and/or captured within the basket 4 before at least portions of the distal end of the basket 4 are withdrawn in a proximal direction. The distal most portion, or ends of the basket may remain substantially stationary (relative to the longitudinal direction) as the basket 4 begins its transition from the expanded position to the collapsed position.

A guide 11 may be connected to the distal end 8 of the sheath 10 to assist in forming the expanded basket 4 as the elongate member 6 and, thus, the legs 22 are moved in the distal direction relative to the sheath 10. The guide 11 may be made from any suitable biocompatible material, including the metals or metal alloys discussed above with respect to the legs 22. The guide 11 may be connected to the sheath 10 in any conventional way. For example, the guide 11 may be threaded and the distal end 8 of the sheath 10 may include corresponding threads (not shown) to facilitate a threaded connection. In an alternate exemplary embodiment, the guide 11 may be press-fit and/or adhered to the sheath 10. The guide 11 may define a plurality of lumens 9 to assist in guiding the legs 22. As shown in, for example, FIGS. 1 and 2, the guide 11 may define at least as many lumens 9 as there are legs 22 of the basket 4, and each leg 22 of the basket 4 may be movably disposed within a respective lumen 9 of the guide 11. In an exemplary embodiment, the guide 11 may be sized and/or otherwise configured to fit substantially completely within the sheath 10. In another exemplary embodiment, however, a portion of the guide 11 may extend beyond the distal end 8.

A joint 13 may be fixedly connected to a distal end of each leg 22 by, for example, weldments, adhesives, solder, threads, and/or any other conventional means. The joint 13 may be made from any suitable biocompatible material, including the metals or alloys discussed above with respect to the legs 22, and the joint 13 may include at least one thru hole sized to accept, in a sliding engagement, a distal length L of an additional leg 22. This provides a relatively loose, moveable connection between the legs 22. As will be discussed in greater detail below, each leg 22 may include a first section 38, a second section 40, and a third section 42, and the distal length L may consist of at least the second section 40 and the third section 42. The joint 13, may be of any size, shape, and/or other configuration to facilitate a movable connection between the distal-most end of a leg 22 and the distal length L of, for example, an adjacent leg 22. For example, the joints 13a, 13b, 13c may be substantially cylindrical. Alternatively, the joints 13a, 13b, 13c may be substantially rectangular or substantially square, and may have rounded, atraumatic edges. In still another exemplary embodiment, the joints 13a, 13b, 13c may be loops formed at the distal end of each respective leg 22a, 22b, 22c. The joints 13a, 13b, 13c may slide along the distal lengths L as the basket 4 is expanded and collapsed. As illustrated in, for example, FIG. 3, the distal-most end of each respective leg 22a, 22b, 22c may terminate at a respective joint 13a, 13b, 13c, and the joints 13a, 13b, 13c may remain substantially outside of the guide 11 when the basket 4 is in the collapsed position (see FIG. 2). In an exemplary embodiment, the joints 13a, 13b, 13c, may work in conjunction with the guide 11 to assist in forming the basket shapes discussed above. In such an embodiment, the joints 13a, 13b, 13c, may also work in conjunction with the guide 11 to expand and collapse the basket 4, in a substantially radial direction, as described above.

As shown in FIGS. 4 and 5, the first section 38 may extend substantially parallel to a longitudinal axis Y of the sheath 10. The longitudinal axis Y may define the center of the lumen 12 of the sheath 10. The first section 38 may remain substantially within the lumen 12 while the basket 4 is in an exemplary fully expanded position. The second section 40 of each leg 22 may be disposed distal the first section 38. The second section 40 may be bent, shaped, formed, or otherwise disposed at a pitch angle A. The pitch angle A may be defined by the second section 40 and a plane X that is perpendicular to the longitudinal axis Y of the sheath 10. The pitch angle A, and, thus, the second section 40 may define the width W of the basket 4. The pitch angle A may be between 0 and 90 degrees and, in an exemplary embodiment, the pitch angle A may be between approximately 30 degrees and approximately 45 degrees.

The second section 40 may also include at least one radius of curvature R. The radius of curvature may assist in defining the height H of the basket 4. In an exemplary embodiment, the pitch angle A and the radius of curvature R of the second section 40 may be achieved through the use of a shape memory material. In such an embodiment, the second section 40 may be substantially straight when the basket 4 is in the collapsed position, and the pitch angle A and the radius of curvature R may take shape once the second section 40 is substantially beyond the distal end 8 of the sheath 10. In addition, the third section 42 may extend substantially perpendicular to the longitudinal axis Y of the sheath 10, and may extend substantially parallel to the plane X. Similar to the first section 38, the third section 42 may be substantially straight. The third section 42 may be connected to, and may terminate at, a respective joint 13.

As illustrated in FIG. 1, at least some aspects of the present disclosure may be used, for example, to retrieve a stone 34, calculus, or other foreign matter or material from any location within the body, such as, for example, in the urinary tract of the patient. The device 2 may be inserted through the urethra or other body cavity of the patient or, alternatively, the device 2 may be inserted percutaneously to a treatment site. The treatment site within the body may correspond to the location of a targeted stone 34.

The stone 34 targeted for retrieval may be a kidney stone, a struvite, a uric acid stone, a cystine stone, or other solid deposit commonly removed from a body structure or passageway within the body. Such stones 34 may contain various combinations of chemicals, including, but not limited to, calcium, oxalate, and phosphate. The stone 34 may be of any size and could have a length or diameter of approximately 1 mm to 12 mm. These lengths and diameters are merely exemplary, and aspects of the present disclosure may assist in the retrieval of stones larger or smaller than those discussed herein. Stones 34 may be of any shape and could be, for example, flat, round, smooth, or jagged. The device 2 may retrieve stones 34 that are both impacted and free floating.

The device 2 may be advanced to the treatment site through an access sheath, stent, or other access or dilatation device known in the art. In addition, the device 2 may be used in conjunction with an endoscope (not shown) or other type of intracorporeal scope known in the art. The endoscope may advance through the body over a guidewire to the treatment site. Alternatively, the endoscope may be independently fed to the treatment site without the use of a guidewire. Once the treatment site has been reached, the device 2 may be fed through an access port of the endoscope to gain access to the stone 34.

While being advanced to the treatment site, the basket 4 of the device 2 may be at least partially, and preferably substantially fully, enclosed within the sheath 10. This configuration (shown in FIG. 2) may minimize the size of the device 2 and may assist in advancing the device 2 through the endoscope. Upon exiting the endoscope and accessing the stone 34, the user may extend at least a portion of the basket 4 from the distal end 8 of the sheath 10.

The basket 4 may expand substantially radially, in an iris-like motion, when transitioning from the collapsed position to the expanded position. The guide 11 will guide the legs 22a, 22b, 22c out of the distal end 8 of the sheath 10 in a desired direction and the joints 13a, 13b, 13c may slide along the distal length L of each adjacent leg 22a, 22b, 22c as the basket 4 transitions to the expanded position. As the joints 13a, 13b, 13c slide along the distal length L, the distal end of each of the legs 22a, 22b, 22c moves in one of a substantially counter-clockwise direction (such as in FIG. 3) and a substantially clockwise direction, as viewed from an open end of the basket. Expansion substantially in the radial direction forms the height H of the basket 4. Expansion substantially in the radial direction also results in minimal expansion in the distal direction, thereby forming the width W of the basket 4. The expanded basket 4 may be manipulated relative to the stone 34 so as to capture the stone 34 within the basket 4. The basket 4 may be substantially open at its distal end 26 to facilitate the capture of the stone 34.

Once the stone 34 is within the basket 4, the user may transition the basket 4 from the expanded position to a substantially collapsed position. The basket 4 may collapse in an iris-like motion. The width W of the basket may remain substantially constant and the height H of the basket 4 may decrease while the basket 4 begins to transition from the expanded position to the collapsed position. Thus, the legs 22a, 22b, 22c may collapse radially around the stone 34 and may make contact with the stone's surface while the extent to which the basket 4 extends distally from the distal end 8 of the sheath 10 does not significantly change. Once captured, the stone 34 may be retrieved by removing the device 2 from the body of the patient.

If the targeted stone 34 is larger than approximately 3 mm, the stone 34 may be too large to be safely removed from the body. In these situations, the user may use the device 2 to capture and assist in immobilizing the stone 34. The user may then perform a stone-reduction process, such as, for example, laser lithotripsy, to break up or otherwise reduce the size of the stone 34. The device 4 may assist in preventing particles of the stone 34 from migrating or escaping from the treatment site during or after the stone-reduction process. A laser fiber (not shown) or other conventional device may be used to break up the stone 34. The laser fiber may be fed through an access port of an endoscope and may be activated and controlled by the user to reduce the size of the stone 34 or to fragment it into smaller pieces. Once the stone 34 has been reduced, the device 2 may act as a sweeping device to sweep stones and stone particles obtained from the reduction process out of the body.

Other embodiments of the invention will be apparent to those skilled in the art from consideration of the specification and practice of the invention disclosed herein. For example, foam, mesh, webbing, or some other material may be attached to a number of the legs 22 to assist in the capture, retrieval, or immobilization of a stone 34. It is intended that the specification and examples be considered as exemplary only, with the true scope and spirit of the invention being indicated by the following claims.

What is claimed is:

1. A medical device, comprising:
a sheath defining a lumen;
an elongate member disposed within the lumen; and
a basket including a plurality of legs connected to the elongate member, wherein a distal end of each of the plurality of legs moves along a length of an adjacent one of the plurality of legs.

2. The medical device of claim 1, wherein at least one of the plurality of legs includes a joint fixedly connected to the distal end of the leg.

3. The medical device of claim 2, wherein the joint is configured to movably connect the at least one leg to the adjacent one of the plurality of legs.

4. The medical device of claim 1, wherein the basket transitions from a collapsed position to an expanded position in a substantially radial direction.

5. The medical device of claim 1, wherein the distal end of at least one of the plurality of legs moves in one of a substantially counter-clockwise direction and a substantially clockwise direction about a longitudinal axis of the sheath, as viewed from an open end of the basket, when the basket is transitioned from a collapsed position to an expanded position.

6. The medical device of claim 1, wherein a distal end of the basket forms a predetermined shape in an expanded position.

7. The medical device of claim 6, wherein the predetermined shape is one of substantially triangular, substantially square, and substantially round.

8. The medical device of claim 1, wherein at least one of the plurality of legs includes a first section, a second section, and a third section, the first section extending substantially parallel to a longitudinal axis of the sheath when the basket is in an expanded position.

9. The medical device of claim 8, wherein the second section is disposed at a pitch angle when the basket is in an expanded position, the pitch angle being defined by the second section and a plane perpendicular to the longitudinal axis of the sheath.

10. The medical device of claim 8, wherein the second section is curved when the basket is in an expanded position.

11. The medical device of claim 8, wherein the second section defines a width of the basket when the basket is in an expanded position.

12. The medical device of claim 8, wherein the third section extends substantially perpendicular to the longitudinal axis of the sheath when the basket is in an expanded position.

13. The medical device of claim 1, wherein a distal end of the basket is substantially open when the basket is in an expanded position.

14. The medical device of claim 13, wherein the distal end of the basket is tipless.

15. The medical device of claim 1, wherein the basket comprises at least three legs.

16. The medical device of claim 1, further including a guide connected to a distal end of the sheath.

17. The medical device of claim 16, wherein at least one of the plurality of legs is movably disposed within a corresponding lumen of the guide.

18. The medical device of claim 1, wherein the plurality of legs define a height and a width of the basket, the basket being configured such that the width of the basket remains substantially constant and the height of the basket decreases during at least an initial portion of the basket transitioning from an expanded position to a collapsed position.

19. A medical device, comprising:
a basket including a plurality of legs defining a height and a width of the basket, the basket being configured such that the width of the basket remains substantially constant and the height of the basket decreases during at least an initial portion of the basket transitioning from an expanded position to a collapsed position.

20. The medical device of claim 19, further including a sheath defining a lumen, wherein at least one of the plurality of legs extends substantially completely beyond a distal end of the sheath in the expanded position.

21. The medical device of claim 20, wherein the at least one leg is disposed substantially completely within the lumen of the sheath in the collapsed position.

22. The medical device of claim 20, wherein a distal end of the at least one leg moves in one of a substantially counter-clockwise direction and a substantially clockwise direction about a longitudinal axis of the sheath, as viewed from an open end of the basket, when the basket is transitioned from the collapsed position to the expanded position.

23. The medical device of claim 20, further including a guide connected to the distal end of the sheath.

24. The medical device of claim 23, wherein the at least one leg is movably disposed within a corresponding lumen of the guide.

25. The medical device of claim 20, wherein the least one leg includes a first section, a second section, and a third section, the first section extending substantially parallel to a longitudinal axis of the sheath when the basket is in an expanded position.

26. The medical device of claim 25, wherein the second section is disposed at a pitch angle when the basket is in an expanded position, the pitch angle being defined by the second section and a plane perpendicular to the longitudinal axis of the sheath.

27. The medical device of claim 25, wherein the second section is curved when the basket is in an expanded position.

28. The medical device of claim 25, wherein the second section defines the width of the basket when the basket is in an expanded position.

29. The medical device of claim 25, wherein the third section extends substantially perpendicular to the longitudinal axis of the sheath when the basket is in an expanded position.

30. The medical device of claim 19, wherein a distal end of the basket is substantially open when the basket is in an expanded position.

31. The medical device of claim 30, wherein the distal end of the basket is tipless.

32. The medical device of claim 19, wherein the basket comprises at least three legs.

33. A method of removing matter from the body of a patient, comprising:
providing a medical device including a sheath defining a lumen, an elongate member disposed within the lumen, and a basket including a plurality of legs connected to the elongate member, wherein a distal end of each of the plurality of legs moves along a length of an adjacent one of the plurality of legs;
advancing the medical device to a treatment site within the body of the patient;
capturing the matter within the basket of the device; and
removing the medical device from the body of the patient.

34. The method of claim 33, wherein the matter is one of a kidney stone, a struvite, a uric acid stone, a cystine stone, and a solid deposit.

35. The method of claim 33, wherein capturing the matter further includes reducing a height of the basket while maintaining a width of the basket substantially constant.

36. The method of claim 33, further including transitioning the basket from a collapsed position to an expanded position.

37. The method of claim 36, wherein transitioning the basket further includes manipulating the distal end of at least one of the plurality of legs in one of a substantially counter-clockwise direction and a substantially clockwise direction about a longitudinal axis of the sheath, as viewed from an open end of the basket.

38. The method of claim 33, wherein transitioning the basket further includes forming a distal end of the basket into a predetermined shape in the expanded position.

39. The method of claim 38, wherein the predetermined shape is one of substantially triangular, substantially square, and substantially round.
